(19) United States
(12) Patent Application Publication
(10) Pub. No.: US 2023/0056604 A1
(43) Pub. Date: Feb. 23, 2023

(54) EPIDERMAL GROWTH FACTOR RECEPTOR TYROSINE KINASE INHIBITORS FOR THE TREATMENT OF CANCER

(71) Applicant: AstraZeneca AB, Sodertalje (SE)

(72) Inventors: Nicolas FLOCH, Cambridge (GB); Paul David SMITH, Cambridge (GB); Matthew Joseph MARTIN, Cambridge (GB)

(21) Appl. No.: 17/759,119

(22) PCT Filed: Jan. 19, 2021
(86) PCT No.: PCT/EP2021/051054

§ 371 Date: Jul. 20, 2022

Related U.S. Application Data
(60) Provisional application No. 62/963,213, filed on Jan. 20, 2020.

(51) Int. Cl. A61K 31/506; A61P 35/00; A61K 31/40

(52) U.S. Cl. CPC A61K 31/506; A61P 35/00; A61K 31/40

(57) ABSTRACT

The specification relates to epidermal growth factor receptor (EGFR) tyrosine kinase inhibitors (TKIs) for use in the treatment of cancer, wherein the EGFR TKI is administered in combination with a Smac mimetic.

### RELATED APPLICATION

This application claims benefit of priority under 35 U.S.C. § 119(e) of the U.S. Provisional Application No. 62/963,213, filed Jan. 20, 2020, which is incorporated by reference herein in its entirety for all purposes.

### FIELD

The specification relates to an Epidermal Growth Factor Receptor (EGFR) Tyrosine Kinase Inhibitor (TKI) for use in the treatment of cancer, wherein the EGFR TKI is administered in combination with a Smac mimetic.

### BACKGROUND

The discovery of activating mutations in the epidermal growth factor receptor (EGFR) has revolutionized the treatment of the disease. In 2004, it was reported that activating mutations in exons 18-21 of EGFR correlated with a response to EGFR-TKI therapy in NSCLC (Science [2004], vol. 304, 1497-1500; New England Journal of Medicine [2004], vol. 350, 2129-2139). It is estimated that these mutations are prevalent in approximately 10-16% of NSCLC human patients in the United States and Europe, and in approximately 30-50% of NSCLC human patients in Asia. Two of the most significant EGFR activating mutations are exon 19 deletions and missense mutations in exon 21. Exon 19 deletions account for approximately 45% of known EGFR mutations. Eleven different mutations, resulting in deletion of three to seven amino acids, have been detected in exon 19, all centred around the uniformly deleted codons for amino acids 747-749. The most significant exon 19 deletion is E746-A750. The missense mutations in exon 21 account for approximately 39-45% of known EGFR mutations, of which the substitution mutation L858R accounts for approximately 39% of the total mutations in exon 21 (J. Thorac. Oncol. [2010], 1551-1558).

Two first generation (erlotinib & gefitinib), two second generation (afatinib & dacomitinib) and a third generation (osimertinib) epidermal growth factor receptor (EGFR) tyrosine kinase inhibitors (TKIs) are currently available for the management of EGFR mutation-positive NSCLC. All these TKIs are effective in patients with NSCLC whose tumours harbour the in-frame deletions in exon 19 and the L858R point mutation in exon 21. These two mutations represent approximatively 90% of all EGFR mutations. In approximately 50% of patients, resistance to first- and second-generation EGFR TKI is mediated by the acquisition of the ‘gatekeeper’ mutation T790M. Currently, osimertinib is the only registered EGFR TKI that is active against exon 19 deletions and L858R mutation, regardless of the presence of T790M mutation. However, even patients treated with osimertinib ultimately progress, predominantly due to the development of acquired resistance resulting from other resistance mechanisms. As such, there remains a need to develop new therapies for the treatment of NSCLC, especially for patients whose disease has progressed following treatment with a third generation EGFR TKI.

Induction of programmed cell death via apoptosis is a critical mechanism of the anticancer effects of osimertinib and other EGFR TKIs. Apoptosis can be activated via intracellular signalling (the so-called “intrinsic” apoptotic pathway) or via signals activated by extracellular ligands (the “extrinsic” pathway). Both the c-IAP (IAP=“inhibitor of apoptosis proteins”) and x-IAP proteins are key regulators of extrinsic apoptosis, acting to prevent its triggering. Several small molecule inhibitors known as Smac mimetics have been developed that directly bind both c-IAP and x-IAP to inhibit their function, leading the execution of apoptosis.

### SUMMARY

The present specification provides a means for enhancing the anti-proliferative and pro-apoptotic effects of EGFR TKI treatment in NSCLC, utilising Smac mimetic compounds in combination with EGFR TKIs.

Through laboratory experiments with populations of cancer cells sensitive to osimertinib, it has been found that the effects of EGFR TKIs may be enhanced in some patients by the use of Smac mimetics.

It has also been found that a combination of EGFR TKI and Smac mimetics may provide an effective first-line therapy against EGFR-associated cancer, i.e. in patients who have not received previous treatment with an EGFR TKI (referred to herein as EGFR TKI-naïve patients). In such patients, the combination treatment may act to delay or prevent development of resistance.

Furthermore, it has been found that the subset of cells that survive EGFR TKI treatment but exist in a non-proliferative pre-resistant state (herein referred to as Drug Tolerant Persister [DTP] cells), upregulate c-IAP1 and c-IAP2 and accordingly are sensitive to Smac mimetic, and that treatment with these agents resulted in cell death.

Without being bound by theory, it is proposed that, in cancer cells reliant on the EGFR pathway, inhibition of this protein induces a state in which cells are susceptible to Smac mimetics. Cells that survive chronic treatment with EGFR TKI monotherapy, have a defect in cell death and can act as a reservoir for the development of clinical resistance. However, in a subset of these patients, cellular adaptations required by cancer cells to avoid death in the presence of EGFR inhibition may uncover a novel vulnerability to Smac mimetics. In preclinical cell line models, a subset of cells tolerant to osimertinib showed enhanced sensitivity to Smac mimetics compared to osimertinib-sensitive parental cells, either in the absence or presence of co-dosed osimertinib. Smac mimetics induced a significant level of apoptosis in DTP cells at doses that did not affect parental cells. Tolerant cells which display enhanced sensitivity to Smac mimetics demonstrated an upregulation of the mRNA corresponding to both the c-IAP1 and c-IAP2 protein. Therefore, a high expression of these mRNA or protein markers in patient tumour tissue may be a potential biomarker for sensitivity to Smac mimetics in patients.

This specification thus discloses a combination of an EGFR TKI and a Smac mimetic both as a first-line treatment (i.e. in EGFR TKI-naïve patients) and as a treatment at the stage of minimal residual disease (i.e. in patients previously treated with EGFR TKIs, where combination treatment is initiated at the point of maximal drug response) of EGFR-mutant NSCLC.

In a first aspect, there is provided an EGFR TKI for use in the treatment of cancer in a human patient, wherein the EGFR TKI is administered in combination with a Smac mimetic.

In a further aspect, there is provided a method of treating cancer in a human patient in need of such a treatment, comprising administering to the human patient a therapeutically effective amount of an EGFR TKI, wherein the EGFR TKI is administered in combination with a therapeutically effective amount of a Smac mimetic.

In a further aspect, there is provided the use of an EGFR TKI in the manufacture of a medicament for the treatment of cancer in a human patient, wherein the EGFR TKI is administered in combination with a Smac mimetic.

In a further aspect, there is provided a pharmaceutical composition comprising an EGFR TKI, a Smac mimetic and a pharmaceutically acceptable diluent or carrier.

In a further aspect, there is provided a Smac mimetic for use in the treatment of non-small cell lung cancer in a human patient, wherein the patient's disease has reached maximal response during or after previous EGFR TKI treatment.

### DESCRIPTION OF FIGURES

FIG. 1: A subset of EGFRm NSCLC cell lines upregulate the expression of c-IAP1 and c-IAP2 mRNA after prolonged treatment with osimertinib. RNA sequencing (RNAseq) was performed in cells chronically treated with osimertinib (14 days) and compared to untreated (DMSO) or acutely treated (24 h) cells. Levels of BIRC2 mRNA (c-IAP1) and BIC3 mRNA (c-IAP2) were plotted on a log 2 scale.

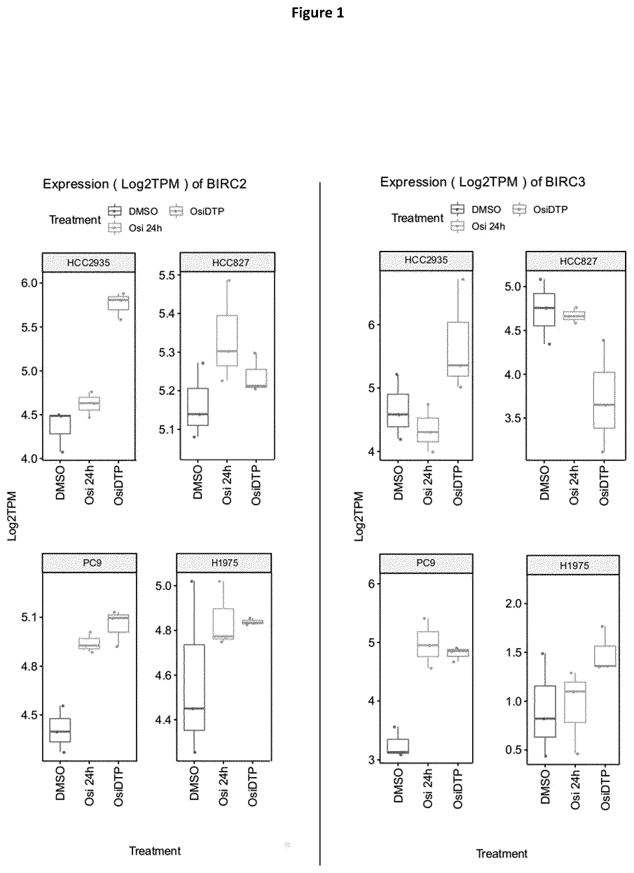

FIG. 2: Smac mimetic AZD5582 enhances osimertinib-induced apoptosis in a panel of EGFRm cell lines Caspase-3/7 activation, a direct readout of apoptotic initiation was measured after 48 h of treatment with either osimertinib monotherapy or its combination with AZD5582 in a panel of 6 EGFRm cell lines. Data was calculated as the number of apoptotic events divided by cell confluence, and normalised to the values for DMSO control. Data are presented on a log scale to better visualize all cell lines.

FIG. 3: Multiple Smac mimetic molecules enhance osimertinib-induced apoptosis in NCI-H1975 and PC9 cells Caspase-3/7 activation, a direct readout of apoptotic initiation was measured after 48 h of treatment with either osimertinib monotherapy or its combination with 4 distinct Smac mimetic small molecules in NCI-H1975 and PC9 cells. Data was calculated as the number of apoptotic events divided by cell confluence, and normalised to the values for DMSO control.

FIG. 4: AZD5582 enhances the antiproliferative effects of osimertinib in a range of EGFRm cell lines. HCC2935, NCI-H1975 and PC9 cells were treated with osimertinib, AZD5582 or the combination of the two agents for 10 days, after which time drug was removed to allow regrowth of the cells. Cell confluence was measured on the Incucyte imaging platform as a surrogate for cell number.

FIG. 5: Cells treated with the osimertinib/AZD5582 combination fail to regrow after drug removal. Representative images were taken from the cell growth experiments in PC9 and HCC2935 cell lines described in FIG. 4. Cells treated for 10 days with either osimertinib alone or osimertinib in combination with AZD5582, at which time drug was removed from 7 days.

FIG. 6: osimertinib DTPs are sensitive to Smac mimetic treatment. Parental PC9 cells were treated with the combination of osimertinib and 4 distinct Smac mimetics to determine the rate of DTP survival and re-growth. Cell confluence was measured on the Incucyte imaging platform as a surrogate for DTP number.

FIG. 7: Smac mimetic treatment induces apoptosis in DTPs. PC9 DTPs were generated by treatment with osimertinib monotherapy for 14 days, followed by treatment with osimertinib in combination with Smac mimetics for 72 h. Cells were co-treated with a green fluorescent caspase activity reagent and monitored over time on the Incucyte imaging platform.

FIG. 8: AZD5582 enhances the antiproliferative effects of osimertinib in PC9 xenograft in vivo. Tumour growth inhibition following dosing of vehicle, osimertinib 25 mg/kg PO QD, AZD5582 2 mg/kg IV QW, or the combination of the two agents for 3 weeks followed by a period of re-growth in the subcutaneous, PC9 model in nude mice. Data are represented as mean ±SEM (n=8 per group) or as tumour volume of individual mouse.

FIG. 9: AZD5582 delivered at the time of minimal residual disease enhances the antiproliferative effects of osimertinib in PC9 xenograft in vivo. Tumour growth inhibition following dosing of vehicle for 3 weeks, osimertinib 25 mg/kg PO QD for 6 weeks, or osimertinib 25 mg/kg PO QD for 3 weeks followed by the combination of osimertinib 25 mg/kg PO QD and AZD5582 2 mg/kg IV QW for 3 weeks followed by a period of re-growth in the subcutaneous, PC9 model in nude mice. Data are represented as mean±SEM (n=8 per group) or as tumour volume of individual mouse.

### DETAILED DESCRIPTION

EGFR Mutation Positive NSCLC and Diagnostic Methods

In embodiments, the cancer is lung cancer, such as non-small cell lung cancer (NSCLC).

In embodiments, the cancer upregulates IAP. In embodiments, the cancer overexpresses IAP. In embodiments, the cancer has increased expression of IAP. In embodiments, the cancer has increased expression of IAP as a result of exposure to an EGFR TKI.

In embodiments, the NSCLC is an EGFR mutation-positive NSCLC.

In embodiments, the EGFR mutation-positive NSCLC comprises activating mutations in EGFR. In further embodiments, the EGFR mutation-positive NSCLC comprises non-resistant mutations. In further embodiments, the activating mutations in EGFR comprise activating mutations in exons 18-21. In further embodiments, the activating mutations in EGFR comprise exon 19 deletions or missense mutations in exon 21. In further embodiments, the activating mutations in EGFR comprise exon 19 deletions or L858R substitution mutations. In further embodiments, the mutations in EGFR comprise a T790M mutation.

In embodiments, the EGFR mutation-positive NSCLC is a locally advanced EGFR mutation-positive NSCLC.

In embodiments, the EGFR mutation-positive NSCLC is a metastatic EGFR mutation-positive NSCLC.

In embodiments, the EGFR mutation-positive NSCLC is not amenable to curative surgery or radiotherapy.

There are numerous methods to detect EGFR activating mutations, of which the skilled person will be aware. A number of tests suitable for use in these methods have been approved by the US Food and Drug Administration (FDA). These include both tumour tissue and plasma based diagnostic methods. In general, the EGFR mutation status is first assessed using a tumour tissue biopsy sample derived from the human patient. If a tumour sample is unavailable, or if the tumour sample is negative, the EGFR mutation status may be assessed using a plasma sample. A particular example of a suitable diagnostic test to detect EGFR mutations, and in particular to detect exon 19 deletions, L858R substitution mutations and the T790M mutation, is the Cobas™ EGFR Mutation Test v2 (Roche Molecular Diagnostics).

In embodiments, therefore, the EGFR mutation-positive NSCLC comprises activating mutations in EGFR (such as activating mutations in exons 18-21, for example exon 19 deletions, missense mutations in exon 21, and L858R substitution mutations; and resistance mutations such as the T790M mutation), wherein the EGFR mutation status of the human patient has been determined using an appropriate diagnostic test. In further embodiments, the EGFR mutation status has been determined using a tumour tissue sample. In further embodiments, the EGFR mutation status has been determined using a plasma sample. In further embodiments, the diagnostic method uses an FDA-approved test. In further embodiments, the diagnostic method uses the Cobas™ EGFR Mutation Test (v1 or v2).

In embodiments, the human patient is an EGFR TKI-naïve human patient.

In embodiments the human patient has previously received EGFR TKI treatment. In embodiments the human patient has previously been treated with osimertinib or a pharmaceutically acceptable salt thereof. In further embodiments, the human patient's disease has reached the stage of maximal response (minimal residual disease) during or after previous EGFR TKI treatment. In further embodiments, the human patient's disease has reached maximal response during or after previous treatment with osimertinib or a pharmaceutically acceptable salt thereof. EGFR TKI treatment includes treatment with either a first-, second- or third-generation EGFR TKI or combinations thereof. In embodiments, the human patient has developed EGFR T790M mutation-positive NSCLC.

In embodiments, the administration of EGFR TKI in combination with a Smac mimetic induces cell death in drug tolerant persister cells.

EGFR TKIs

EGFR TKIs can be characterised as either first-, second- or third-generation EGFR TKIs, as set out below.

First-generation EGFR TKIs are reversible inhibitors of EGFR bearing activating mutations that do not significantly inhibit EGFR bearing a T790M mutation. Examples of first-generation TKIs include gefitinib and erlotinib.

Second-generation EGFR TKIs are irreversible inhibitors of EGFR bearing activating mutations that do not significantly inhibit EGFR bearing the T790M mutation. Examples of second-generation TKIs include afatinib and dacomitinib.

Third-generation EGFR TKIs are inhibitors of EGFR bearing activating mutations that also significantly inhibit EGFR bearing the T790M mutation and do not significantly inhibit wild-type EGFR. Examples of third generation TKIs include compounds of formula (I), osimertinib, AZD3759, lazertinib, nazartinib, CO1686 (rociletinib), HM61713, ASP8273, EGF816, PF-06747775 (mavelertinib), avitinib (abivertinib), alflutinib (AST2818) and CX-101 (RX-518), almonertinib (HS-10296) and BPI-7711.

In embodiments, the EGFR TKI is a first-generation EGFR TKI. In further embodiments, the first-generation EGFR TKI is selected from the group consisting of gefitinib or a pharmaceutically acceptable salt thereof, icotinib or a pharmaceutically acceptable salt thereof, and erlotinib or a pharmaceutically acceptable salt thereof.

In embodiments, the EGFR TKI is a second-generation EGFR TKI. In further embodiments, the second-generation EGFR TKI is selected from dacomitinib, or a pharmaceutically acceptable salt thereof and afatinib or a pharmaceutically acceptable salt thereof.

In embodiments, the EGFR TKI is a third-generation EGFR TKI. In a further embodiment, the third-generation EGFR TKI is a compound of formula (I), as defined below. In further embodiments, the third-generation EGFR TKI is selected from the group consisting of osimertinib or a pharmaceutically acceptable salt thereof, AZD3759 or a pharmaceutically acceptable salt thereof, lazertinib or a pharmaceutically acceptable salt thereof, abivertinib or a pharmaceutically acceptable salt thereof, alflutinib or a pharmaceutically acceptable salt thereof, CX-101 or a pharmaceutically acceptable salt thereof, HS-10296 or a pharmaceutically acceptable salt thereof and BPI-7711 or a pharmaceutically acceptable salt thereof. In further embodiments, the third generation EGFR TKI is osimertinib or a pharmaceutically acceptable salt thereof.

Compounds of Formula (I)

In an aspect, the EGFR TKI is a compound of Formula (I):

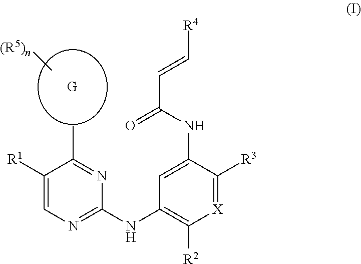

wherein:

G is selected from 4,5,6,7-tetrahydropyrazolo[1,5-a]pyridin-3-yl, indol-3-yl, indazol-1-yl, 3,4-dihydro-1H-[1,4]oxazino[4,3-a]indol-10-yl, 6,7,8,9-tetrahydropyrido[1,2-a]indol-10-yl, 5,6-dihydro-4H-pyrrolo[3,2,1-ij]quinolin-1-yl, pyrrolo[3,2-b]pyridin-3-yl and pyrazolo[1,5-a]pyridin-3-yl;

R¹ is selected from hydrogen, fluoro, chloro, methyl and cyano;

R² is selected from methoxy, trifluoromethoxy, ethoxy, 2,2,2-trifluoroethoxy and methyl;

R³ is selected from (3R)-3-(dimethylamino)pyrrolidin-1-yl, (3S)-3-(dimethyl-amino)pyrrolidin-1-yl, 3-(dimethylamino)azetidin-1-yl, [2-(dimethylamino)ethyl]-(methyl)amino, [2-(methylamino)ethyl](methyl)amino, 2-(dimethylamino)ethoxy, 2-(methylamino)ethoxy, 5-methyl-2,5-diazaspiro[3,4]oct-2-yl, (3aR,6aR)-5-methylhexa-hydro-pyrrolo[3,4-b]pyrrol-1(2H)-yl, 1-methyl-1,2,3,6-tetrahydropyridin-4-yl, 4-methylpiperizin-1-yl, 4-[2-(dimethylamino)-2-oxoethyl]piperazin-1-yl, methyl[2-(4-methylpiperazin-1-yl)ethyl]amino, methyl[2-(morpholin-4-yl)ethyl]amino, 1-amino-1,2,3,6-tetrahydropyridin-4-yl and 4-[(2S)-2-aminopropanoyl]piperazin-1-yl;

R⁴ is selected from hydrogen, 1-piperidinomethyl and N,N-dimethylaminomethyl;

R⁵ is independently selected from methyl, ethyl, propyl, 2,2-difluoroethyl, 2,2,2-trifluoroethyl, fluoro, chloro and cyclopropyl;

X is CH or N; and

n is 0, 1 or 2;

or a pharmaceutically acceptable salt thereof.

In a further aspect there is provided a compound of Formula (I), as defined above, wherein G is selected from indol-3-yl and indazol-1-yl; R¹ is selected from hydrogen, fluoro, chloro, methyl and cyano; R² is selected from methoxy and 2,2,2-trifluoroethoxy; R³ is selected from [2-(dimethylamino)ethyl]-(methyl)amino, [2-(methylamino)ethyl](methyl)amino, 2-(dimethylamino)ethoxy and 2-(methylamino)ethoxy; R⁴ is hydrogen; R⁵ is selected from methyl, 2,2,2-trifluoroethyl and cyclopropyl; X is CH or N; and n is 0 or 1; or a pharmaceutically acceptable salt thereof.

Examples of compounds of Formula (I) include those described in WO 2013/014448, WO 2015/175632, WO 2016/054987, WO 2016/015453, WO 2016/094821, WO 2016/070816 and WO 2016/173438.

Osimertinib and Pharmaceutical Compositions Thereof

Osimertinib has the following chemical structure:

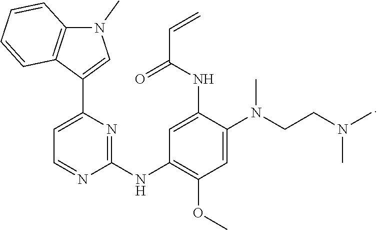 [SMILES: C=CC(=O)Nc1cc(Nc2nccc(-c3cn(C)c4ccccc34)n2)c(OC)cc1N(C)CCN(C)C]

The free base of osimertinib is known by the chemical name: N-(2-{2-dimethylamino ethyl-methylamino}-4-methoxy-5-{[4-(1-methylindol-3-yl)pyrimidin-2-yl]amino}phenyl) prop-2-enamide. osimertinib is described in WO 2013/014448. Osimertinib is also known as AZD9291.

Osimertinib may be found in the form of the mesylate salt: N-(2-{2-dimethylamino ethyl-methylamino}-4-methoxy-5-{[4-(1-methylindol-3-yl)pyrimidin-2-yl]amino}phenyl) prop-2-enamide mesylate salt. Osimertinib mesylate is also known as TAGRISSO™.

Osimertinib mesylate is currently approved as an oral once daily tablet formulation, at a dose of 80 mg (expressed as free base, equivalent to 95.4 mg osimertinib mesylate), for the treatment of metastatic EGFR T790M mutation positive NSCLC human patients. A 40 mg oral once daily tablet formulation (expressed as free base, equivalent to 47.7 mg osimertinib mesylate) is available should dose modification be required. The tablet core comprises pharmaceutical diluents (such as mannitol and microcrystalline cellulose), disintegrants (such as low-substituted hydroxypropyl cellulose) and lubricants (such as sodium stearyl fumarate). The tablet formulation is described in WO 2015/101791.

In embodiments, therefore, osimertinib or a pharmaceutically acceptable salt thereof, is in the form of the mesylate salt, i.e. N-(2-{2-dimethylamino ethyl-methylamino}-4-methoxy-5-{[4-(1-methylindol-3-yl)pyrimidin-2-yl]amino}phenyl) prop-2-enamide mesylate salt.

In embodiments, osimertinib or a pharmaceutically acceptable salt thereof, is administered once daily. In further embodiments, osimertinib mesylate is administered once daily.

In embodiments, the total daily dose of osimertinib is about 80 mg. In further embodiments, the total daily dose of osimertinib mesylate is about 95.4 mg.

In embodiments, the total daily dose of osimertinib is about 40 mg. In further embodiments, the total daily dose of osimertinib mesylate is about 47.7 mg.

In embodiments, osimertinib or a pharmaceutically acceptable salt thereof, is in tablet form.

In embodiments, osimertinib or a pharmaceutically acceptable salt thereof, is administered in the form of a pharmaceutical composition comprising one or more pharmaceutically acceptable excipients. In further embodiments, the composition comprises one or more pharmaceutical diluents (such as mannitol and microcrystalline cellulose), one or more pharmaceutical disintegrants (such as low-substituted hydroxypropyl cellulose) or one or more pharmaceutical lubricants (such as sodium stearyl fumarate).

In embodiments, the composition is in the form of a tablet, wherein the tablet core comprises: (a) from 2 to 70 parts of osimertinib or a pharmaceutically acceptable salt thereof; (b) from 5 to 96 parts of two or more pharmaceutical diluents; (c) from 2 to 15 parts of one or more pharmaceutical disintegrants; and (d) from 0.5 to 3 parts of one or more pharmaceutical lubricants; and wherein all parts are by weight and the sum of the parts (a)+(b)+(c)+(d)=100.

In embodiments, the composition is in the form of a tablet, wherein the tablet core comprises: (a) from 7 to 25 parts of osimertinib or a pharmaceutically acceptable salt thereof; (b) from 55 to 85 parts of two or more pharmaceutical diluents, wherein the pharmaceutical diluents comprise microcrystalline cellulose and mannitol; (c) from 2 to 8 parts of pharmaceutical disintegrant, wherein the pharmaceutical disintegrant comprises low-substituted hydroxypropyl cellulose; (d) from 1.5 to 2.5 parts of pharmaceutical lubricant, wherein the pharmaceutical lubricant comprises sodium stearyl fumarate; and wherein all parts are by weight and the sum of the parts (a)+(b)+(c)+(d)=100.

In embodiments, the composition is in the form of a tablet, wherein the tablet core comprises: (a) about 19 parts of osimertinib mesylate; (b) about 59 parts of mannitol; (c) about 15 parts of microcrystalline cellulose; (d) about 5 parts of low-substituted hydroxypropyl cellulose; and (e) about 2 parts of sodium stearyl fumarate; and wherein all parts are by weight and the sum of the parts (a)+(b)+(c)+(d)+(e)=100.

AZD3759

AZD3759 has the following chemical structure:

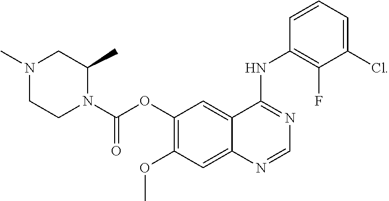 [SMILES: COc1cc2ncnc(Nc3cccc(Cl)c3F)c2cc1OC(=O)N1CCN(C)C[C@H]1C]

The free base of AZD3759 is known by the chemical name: 4-[(3-chloro-2-fluorophenyl)amino]-7-methoxy-6-quinazolinyl (2R)-2,4-dimethyl-1-piperazinecarboxylate. AZD3759 is described in WO 2014/135876.

In embodiments, AZD3759 or a pharmaceutically acceptable salt thereof, is administered twice daily. In further embodiments, AZD3759 is administered twice daily.

In embodiments, the total daily dose of AZD3759 is about 400 mg. In further embodiments, about 200 mg of AZD3759 is administered twice a day.

Lazertinib

Lazertinib has the following chemical structure:

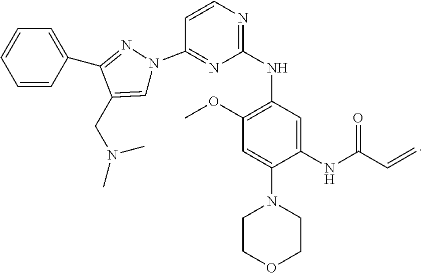 [SMILES: C=CC(=O)Nc1cc(Nc2nccc(-n3cc(CN(C)C)c(-c4ccccc4)n3)n2)c(OC)cc1N1CCOCC1]

The free base of lazertinib is known by the chemical name: N-{5-[(4-{4-[(dimethylamino)methyl]-3-phenyl-1H-pyrazol-1-yl}-2-pyrimidinyl)amino]-4-methoxy-2-(4-morpholinyl)phenyl}acrylamide. Lazertinib is described in WO 2016/060443. Lazertinib is also known by the names YH25448 and GNS-1480.

In embodiments, lazertinib or a pharmaceutically acceptable salt thereof, is administered once daily. In further embodiments, lazertinib is administered once daily.

In embodiments, the total daily dose of lazertinib is about 20 to 320 mg.

In embodiments, the total daily dose of lazertinib is about 240 mg.

Avitinib

Avitinib has the following chemical structure:

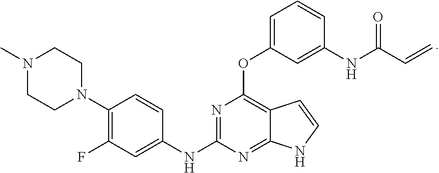 [SMILES: C=CC(=O)Nc1cccc(Oc2nc(Nc3ccc(N4CCN(C)CC4)c(F)c3)nc3[nH]ccc23)c1]

The free base of avitinib is known by the chemical name: N-(3-((2-((3-fluoro-4-(4-methylpiperazin-1-yl)phenyl)amino)-7H-pyrrolo(2,3-d)pyrimidin-4-yl)oxy)phenyl)prop-2-enamide. Avitinib is disclosed in US2014038940. Avitinib is also known as abivertinib.

In embodiments, avitinib or a pharmaceutically acceptable salt thereof, is administered twice daily. In further embodiments, avitinib maleate is administered twice daily.

In embodiments, the total daily dose of avitinib maleate is about 600 mg.

Alflutinib

Alflutinib has the following chemical structure:

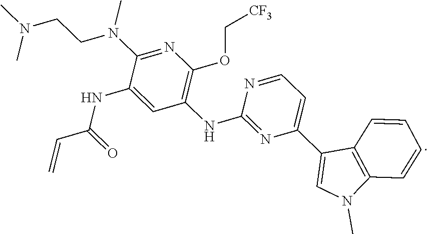 [SMILES: C=CC(=O)Nc1cc(Nc2nccc(-c3cn(C)c4ccccc34)n2)c(OCC(F)(F)F)nc1N(C)CCN(C)C]

The free base of alflutinib is known by the chemical name: N-{2-{[2-(dimethylamino)ethyl](methyl)amino}-6-(2,2,2-trifluoroethoxyl)-5-{[4-(1-methyl-1H -indol-3-yl)pyrimidin-2-yl]amino}pyridin-3-yl}acrylamide. Alflutinib is disclosed in WO 2016/15453. Alflutinib is also known as AST2818.

In embodiments, alflutinib or a pharmaceutically acceptable salt thereof, is administered once daily. In further embodiments, alflutinib mesylate is administered once daily.

In embodiments, the total daily dose of alflutinib mesylate is about 80 mg.

In embodiments, the total daily dose of alflutinib mesylate is about 40 mg.

Afatinib

Afatinib has the following chemical structure:

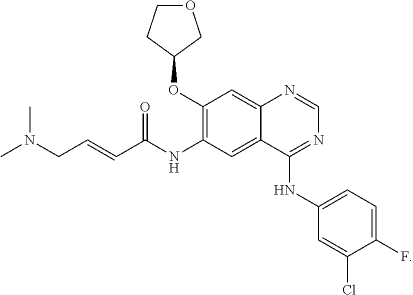 [SMILES: CN(C)C/C=C/C(=O)Nc1cc2c(Nc3ccc(F)c(Cl)c3)ncnc2cc1O[C@H]1CCOC1]

The free base of afatinib is known by the chemical name: N-[4-(3-chloro-4-fluoroanilino)-7-[(3S)-oxolan-3-yl] oxyquinazolin-6-yl]-4-(dimethylamino)but-2-enamide. Afatinib is disclosed in WO 02/50043. Afatinib is also known as Gilotrif.

In embodiments, afatinib or a pharmaceutically acceptable salt thereof, is administered once daily. In further embodiments, afatinib dimaleate is administered once daily.

In embodiments, the total daily dose of afatinib dimaleate is about 40 mg.

In embodiments, the total daily dose of afatinib dimaleate is about 30 mg.

CX-101

CX-101 has the following chemical structure:

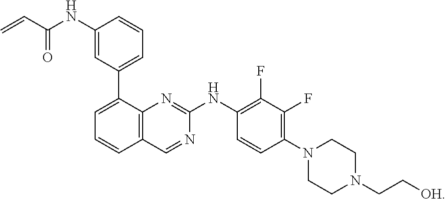 [SMILES: C=CC(=O)Nc1cccc(-c2cccc3cnc(Nc4ccc(N5CCN(CCO)CC5)c(F)c4F)nc23)c1]

The free base of CX-101 is known by the chemical name: N-(3-(2-((2,3-difluoro-4-(4-(2-hydroxyethyl)piperazin-1-yl)phenyl)amino)quinazolin-8-yl)phenyl)acrylamide. CX-101 is disclosed in WO 2015/027222. CX-101 is also known as RX-518.

HS-10296 (Almonertinib)

HS-10296 (almonertinib) has the following chemical structure:

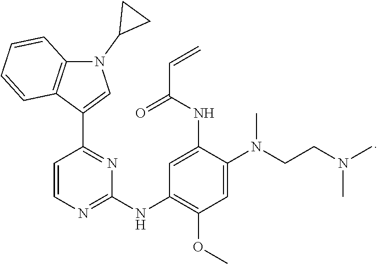 [SMILES: C=CC(=O)Nc1cc(Nc2nccc(-c3cn(C4CC4)c4ccccc34)n2)c(OC)cc1N(C)CCN(C)C]

The free base of HS-10296 is known by the chemical name: N-[5-[[4-(1-cyclopropylindol-3-yl)pyrimidin-2-yl]amino]-2-[2-(dimethylamino)ethyl-methyl-amino]-4-methoxy-phenyl]prop-2-enamide. HS-10296 is disclosed in WO 2016/054987.

In embodiments, the total daily dose of HS-10296 is about 110 mg.

Icotinib

Icotinib has the following chemical structure:

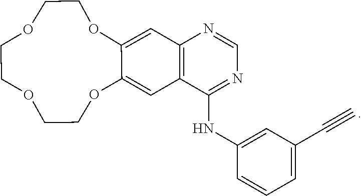 [SMILES: C#Cc1cccc(Nc2ncnc3cc4c(cc23)OCCOCCOCCO4)c1]

The free base of icotinib is known by the chemical name: N-(3-ethynylphenyl)-2,5,8,11-tetraoxa-15,17-diazatricyclo[10.8.0.0^{14,19}]icosa-1(12),13,15,17,19-pentaen-18-amine. Icotinib is disclosed in WO2013064128. Icotinib is also known as Conmana.

In embodiments, icotinib or a pharmaceutically acceptable salt thereof, is administered three times daily. In further embodiments, icotinib hydrochloride is administered three times daily.

In embodiments, the total daily dose of icotinib hydrochloride is about 375 mg.

BPI-7711

BPI-7711 has the following chemical structure:

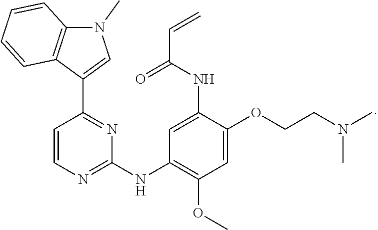 [SMILES: C=CC(=O)Nc1cc(Nc2nccc(-c3cn(C)c4ccccc34)n2)c(OC)cc1OCCN(C)C]

The free base of BPI-7711 is known by the chemical name: N-[2-[2-(dimethylamino)ethoxy]-4-methoxy-5-[[4-(1-methylindol-3-yl)pyrimidin-2-yl]amino]phenyl]prop-2-enamide. BPI-7711 is disclosed in WO 2016/94821.

In embodiments, the total daily dose of BPI-7711 is about 180 mg.

Dacomitinib

Dacomitinib has the following chemical structure:

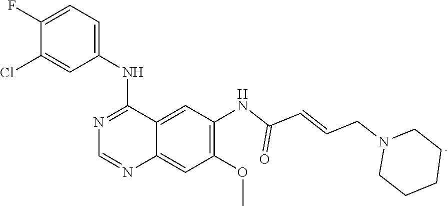 [SMILES: COc1cc2ncnc(Nc3ccc(F)c(Cl)c3)c2cc1NC(=O)/C=C/CN1CCCCC1]

The free form of dacomitinib is known by the chemical name: (2E)-N-{4-[(3-chloro-4-fluorophenyl)amino]-7-methoxyquinazolin-6-yl}-4-(piperidin-1-yl)but-2-enamide. Dacomitinib is disclosed in WO 2005/107758. Dacomitinib is also known by the name PF-00299804.

Dacomitinib may be found in the form of dacomitinib monohydrate, i.e. (2E)-N-{4-[(3-chloro-4-fluorophenyl)amino]-7-methoxyquinazolin-6-yl}-4-(piperidin-1-yl)but-2-enamide monohydrate.

In embodiments, dacomitinib or a pharmaceutically acceptable salt thereof, is administered once daily. In further embodiments, dacomitinib monohydrate is administered once daily.

In embodiments, the total daily dose of dacomitinib monohydrate is about 45 mg.

In embodiments, dacomitinib or a pharmaceutically acceptable salt thereof, is in tablet form.

In embodiments, dacomitinib or a pharmaceutically acceptable salt thereof, is administered in the form of a pharmaceutical composition comprising one or more pharmaceutically acceptable excipients. In further embodiments, the one or more pharmaceutically acceptable excipients comprise lactose monohydrate, microcrystalline cellulose, sodium starch glycolate and magnesium stearate.

Gefitinib

Gefitinib has the following chemical structure:

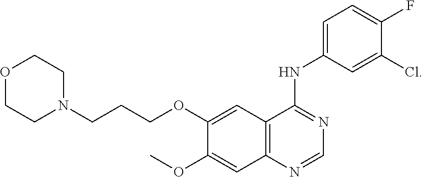 [SMILES: COc1cc2ncnc(Nc3ccc(F)c(Cl)c3)c2cc1OCCCN1CCOCC1]

The free base of gefitinib is known by the chemical name: N-(3-chloro-4-fluorophenyl)-7-methoxy-6-(3-morpholin-4-ylpropoxy)quinazolin-4-amine. Gefitinib is disclosed in WO 1996/033980. Gefitinib is also known as IRESSA™.

In embodiments, gefitinib or a pharmaceutically acceptable salt thereof, is administered once daily. In further embodiments, gefitinib is administered once daily.

In embodiments, the total daily dose of gefitinib is about 250 mg.

Erlotinib

Erlotinib has the following chemical structure:

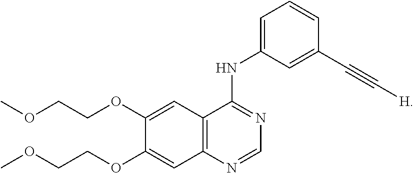

The free base of erlotinib is known by the chemical name: N-(3-ethynylphenyl)-6,7-bis(2-methoxyethoxy) quinazolin-4-amine. Erlotinib is disclosed in WO 1996/030347. Erlotinib is also known as TARCEVA™.

In embodiments, erlotinib or a pharmaceutically acceptable salt thereof, is administered once daily. In further embodiments, erlotinib is administered once daily.

In embodiments, the total daily dose of erlotinib is about 150 mg.

In embodiments, the total daily dose of erlotinib is about 100 mg.

Smac Mimetics

In embodiments, the Smac mimetic is any molecule which binds to and inhibits the activity of one or more IAPs, such as cellular IAP (c-IAP, e.g., c-IAP1 or c-IAP2) or X-linked IAP (x-IAP).

In embodiments, the Smac mimetic is any IAP inhibitor described or claimed in the following publications: US20050197403, U.S. Pat. Nos. 7,244,851, 7,309,792, 7,517,906, 7,579,320, 7,547,724, WO2004/007529, WO 2005/069888, WO 2005/069894, WO2005097791, WO 2006/010118, WO 2006/122408, WO 2006/017295, WO 2006/133147, WO 2006/128455, WO2006/091972, WO 2006/020060, WO 2006/014361, WO 2006/097791, WO 2007/021825, WO 2007/106192, WO2007/101347, WO 2008/045905, WO 2008/016893, WO2008/128121, WO2008/128171, WO 2008/134679, WO 2008/073305, WO 2009/060292, WO 2007/104162, WO 2007/130626, WO 2007/131366, WO 2007/136921, WO 2008/014229, WO 2008/014236, WO 2008/014238, WO 2008/014240, WO 2008/134679, WO2009/136290, WO 2008/014236 and WO 2008/144925.

In embodiments, the Smac mimetic is selected from the group consisting of AZD5582 or a pharmaceutically acceptable salt thereof, birinapant or a pharmaceutically acceptable salt thereof, LCL161 or a pharmaceutically acceptable salt thereof, GDC-0152 or a pharmaceutically acceptable salt thereof, GDC-0917 or a pharmaceutically acceptable salt thereof HGS1029 or a pharmaceutically acceptable salt thereof and AT-406 or a pharmaceutically acceptable salt thereof. In further embodiments, the Smac mimetic is AZD5582 or a pharmaceutically acceptable salt thereof. In further embodiments, the Smac mimetic is AZD5582 dihydrochloride. In further embodiments, the Smac mimetic is Birinapant or a pharmaceutically acceptable salt thereof. In further embodiments, the Smac mimetic is LCL161 or a pharmaceutically acceptable salt thereof. In further embodiments, the Smac mimetic is GDC-0152 or a pharmaceutically acceptable salt thereof.

AZD5582

AZD5582 has the following chemical structure:

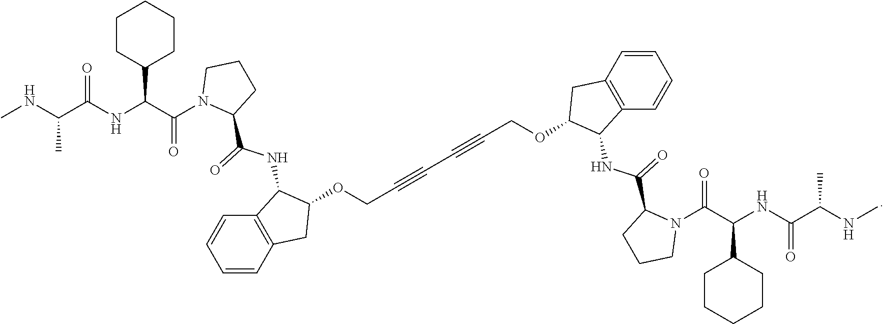

The free base of AZD5582 is known by the chemical name 3,3′-[2,4-Hexadiyne-1,6-diylbis[oxy[(1S,2R)-2,3-dihydro-1H-indene-2,1-diyl]]]bis[N-methyl-L-alanyl-(2S)-2-cyclohexylglycyl-L-prolinamide. AZD5582 is disclosed in WO2010142994.

Birinapant

Birinapant, or TL32711, has the following chemical structure:

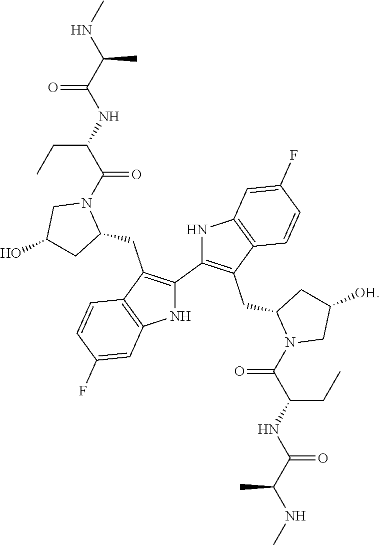 [SMILES: CC[C@H](NC(=O)[C@H](C)NC)C(=O)N1C[C@@H](O)C[C@H]1Cc1c(-c2[nH]c3cc(F)ccc3c2C[C@@H]2C[C@H](O)CN2C(=O)[C@H](CC)NC(=O)[C@H](C)NC)[nH]c2cc(F)ccc12]

The free base of birinapant is known by the chemical name (2S,2′S)—N,N′-[(6,6′-Difluoro-1H,1′H-2,2′-biindole-3,3′-diyl)bis{methylene[(2R,4S)-4-hydroxy-2,1-pyrrolidinediyl][(2S)-1-oxo-1,2-butanediyl]}]bis[2-(methylamino)propanamide]. Birinapant is disclosed in U.S. Pat. No. 8,283,372.

LCL161

LCL161 has the following chemical structure:

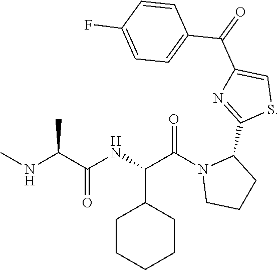 [SMILES: CN[C@@H](C)C(=O)N[C@H](C(=O)N1CCC[C@H]1c1nc(C(=O)c2ccc(F)cc2)cs1)C1CCCCC1]

The free base of LCL161 is known by the chemical name (S)—N—((S)-1-cyclohexyl-2-((S)-2-(4-(4-fluorobenzoyl)thiazol-2-yl)pyrrolidin-1-yl)-2-oxoethyl)-2-(methylamino)propanamide. LCL161 is disclosed in WO2008016893.

GDC-0152

GDC-0152 has the following chemical structure:

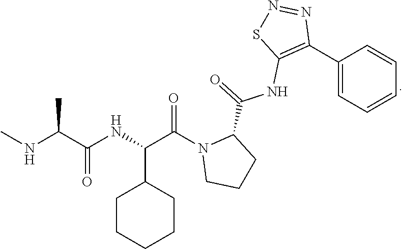 [SMILES: CN[C@@H](C)C(=O)N[C@H](C(=O)N1CCC[C@H]1C(=O)Nc1snnc1-c1ccccc1)C1CCCCC1]

The free base of GDC-0152 is known by the chemical name (S)-1-[(S)-2-cyclohexyl-2-([S]-2-[methylamino]propanamido)acetyl]-N-(4-phenyl-1,2,3-thiadiazol-5-yl)pyrrolidine-2-carboxamide. GDC-0152 is disclosed in US20060014700.

GDC-0917

GDC-0917 has the following chemical structure:

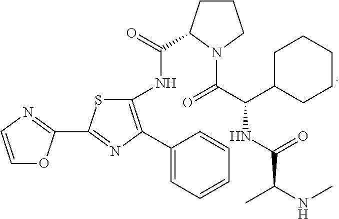 [SMILES: CN[C@@H](C)C(=O)N[C@H](C(=O)N1CCC[C@H]1C(=O)Nc1sc(-c2ncco2)nc1-c1ccccc1)C1CCCCC1]

The free base of GDC-0917 is known by the chemical name (S)-1-((S)-2-cyclohexyl-2-((S)-2-(methylamino)propanamido)acetyl)-N-(2-(oxazol-2-yl)-4-phenylthiazol-5-yl)pyrrolidine-2-carboxamide. GDC-0917 is disclosed in WO2013103703.

AT-406

AT-406 has the following chemical structure:

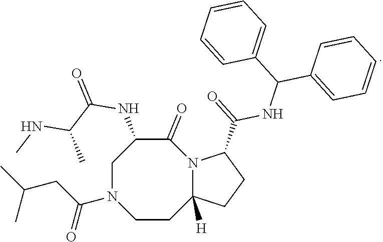 [SMILES: CN[C@@H](C)C(=O)N[C@H]1CN(C(=O)CC(C)C)CC[C@H]2CC[C@@H](C(=O)NC(c3ccccc3)c3ccccc3)N2C1=O]

The free base of AT-406 is known by the chemical name (5S,8S,10aR)—N-benzhydryl-5-((S)-2-(methylamino)propanamido)-3-(3-methylbutanoyl)-6-oxodecahydropyrrolo[l,2-a][l,5]diazocine-8-carboxamide. AT-406 is disclosed in WO2008/128171.

HGS1029

HGS1029 has the following chemical structure:

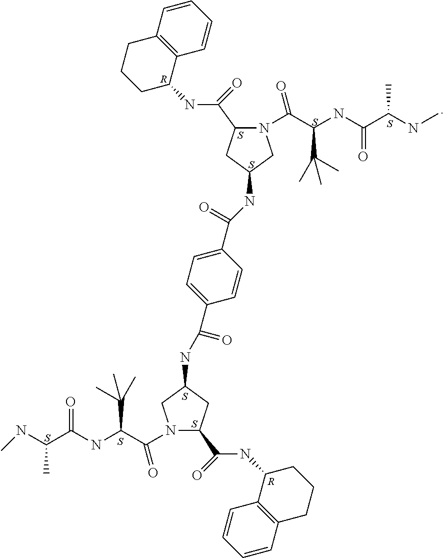

The free base of HGS1029 is known as N1,N4-bis((3S,5S)-1-((S)-3,3-dimethyl-2-((S)-2-(methylamino)propanamido)butanoyl)-5-((R)-1,2,3,4-tetrahydronaphthalen-1-ylcarbamoyl)pyrrolidin-3-yl)terephthalamide. HGS1029 is disclosed in WO2007104162.

### Further Embodiments

In an aspect there is provided an EGFR TKI for use in the treatment of cancer in a human patient, wherein the EGFR TKI is administered in combination with a Smac mimetic. In embodiments, the cancer is lung cancer, such as NSCLC. In yet further embodiments, the NSCLC is an EGFR mutation-positive NSCLC.

In an aspect there is provided a method of treating cancer in a human patient in need of such a treatment comprising administering to the human patient a therapeutically effective amount of an EGFR TKI, wherein the EGFR TKI is administered in combination with a therapeutically effective amount of a Smac mimetic. In embodiments, the cancer is lung cancer, such as NSCLC. In yet further embodiments, the NSCLC is an EGFR mutation-positive NSCLC.

In an aspect there is provided the use of an EGFR TKI in the manufacture of a medicament for the treatment of cancer in a human patient, wherein the EGFR TKI is administered in combination with a Smac mimetic. In embodiments, the cancer is lung cancer, such as NSCLC. In yet further embodiments, the NSCLC is an EGFR mutation-positive NSCLC.

In an aspect there is provided a combination of an EGFR TKI and Smac mimetic for use in the treatment of cancer in a human patient. In embodiments the EGFR TKI is osimertinib or a pharmaceutically acceptable salt thereof. In further embodiments, the human patient is an EGFR TKI-naïve human patient. In further embodiments, the human patient has previously received EGFR TKI treatment. In further embodiments, the human patient has previously received osimertinib or a pharmaceutically acceptable salt thereof. In still further embodiments, the cancer is lung cancer, such as NSCLC. In yet further embodiments, the NSCLC is an EGFR mutation-positive NSCLC.

In an aspect there is provided a method of treating cancer in a human patient in need of such a treatment comprising administering to the human patient a combination of a therapeutically effective amount of an EGFR TKI and a therapeutically effective amount of a Smac mimetic. In embodiments, the EGFR TKI is osimertinib or a pharmaceutically acceptable salt thereof. In further embodiments, the human patient is an EGFR TKI-naïve human patient. In further embodiments, the human patient has previously received EGFR TKI treatment. In further embodiments, the human patient has previously received osimertinib or a pharmaceutically acceptable salt thereof. In still further embodiments, the cancer is lung cancer, such as NSCLC. In yet further embodiments, the NSCLC is an EGFR mutation-positive NSCLC.

In an aspect there is provided the use of a combination of an EGFR TKI and Smac mimetic in the manufacture of a medicament for treatment of cancer in a human patient. In embodiments, the EGFR TKI is osimertinib or a pharmaceutically acceptable salt thereof. In further embodiments, the human patient is an EGFR TKI-naïve human patient. In further embodiments, the human patient has previously received EGFR TKI treatment. In further embodiments, the human patient has previously received osimertinib or a pharmaceutically acceptable salt thereof. In still further embodiments, the cancer is lung cancer, such as NSCLC. In yet further embodiments, the NSCLC is an EGFR mutation-positive NSCLC.

In an aspect there is provided a combination of osimertinib or a pharmaceutically acceptable salt thereof and Smac mimetic for use in the treatment of cancer in a human patient, wherein the osimertinib, or pharmaceutically acceptable salt thereof, is administered to the human patient before the Smac mimetic is administered to the human patient. In embodiments, the cancer is lung cancer, such as NSCLC. In yet further embodiments, the NSCLC is an EGFR mutation-positive NSCLC.

In an aspect there is provided a method of treating cancer in a human patient in need of such a treatment comprising administering to the human patient a combination of a therapeutically effective amount of osimertinib or a pharmaceutically acceptable salt thereof and a therapeutically effective amount of a Smac mimetic, wherein the osimertinib, or pharmaceutically acceptable salt thereof, is administered to the human patient before the Smac mimetic is administered to the human patient. In embodiments, the cancer is lung cancer, such as NSCLC. In yet further embodiments, the NSCLC is an EGFR mutation-positive NSCLC.

In an aspect there is provided the use of a combination of osimertinib or a pharmaceutically acceptable salt thereof and a Smac mimetic for the manufacture of a medicament for the treatment of cancer in a human patient, wherein the osimertinib, or pharmaceutically acceptable salt thereof, is administered to the human patient before the Smac mimetic is administered to the human patient. In embodiments, the cancer is lung cancer, such as NSCLC. In yet further embodiments, the NSCLC is an EGFR mutation-positive NSCLC.

In an aspect there is provided an EGFR TKI for use in the treatment of cancer in a human patient, wherein the treatment comprises the separate, sequential, or simultaneous administration of i) the EGFR TKI and ii) Smac mimetic to the human patient. Where treatment is separate or sequential, the interval between the dose of EGFR TKI and the dose of Smac mimetic may be chosen to ensure the production of a combined therapeutic effect.

In embodiments, the administration of the EGFR TKI and the Smac mimetic is sequential and the EGFR TKI is administered prior to the Smac mimetic.

In embodiments, the EGFR TKI is osimertinib or a pharmaceutically acceptable salt thereof. In further embodiments, the human patient is an EGFR TKI-naïve human patient. In further embodiments, the human patient has previously received EGFR TKI treatment. In further embodiments, the human patient has previously received osimertinib or a pharmaceutically acceptable salt thereof. In still further embodiments, the cancer is lung cancer, such as NSCLC. In yet further embodiments, the NSCLC is an EGFR mutation-positive NSCLC.

In an aspect there is provided a method of treating cancer in a human patient in need of such a treatment comprising the separate, sequential, or simultaneous administration of i) a therapeutically effective amount of an EGFR TKI and ii) a therapeutically effective amount of a Smac mimetic to the human patient. In embodiments, the EGFR TKI is osimertinib or a pharmaceutically acceptable salt thereof. In further embodiments, the human patient is an EGFR TKI-naïve human patient. In further embodiments, the human patient has previously received EGFR TKI treatment. In further embodiments, the human patient has previously received osimertinib or a pharmaceutically acceptable salt thereof. In still further embodiments, the cancer is lung cancer, such as NSCLC. In yet further embodiments, the NSCLC is an EGFR mutation-positive NSCLC.

In an aspect there is provided use of an EGFR TKI in the manufacture of a medicament for the treatment of cancer in a human patient, wherein the treatment comprises the separate, sequential, or simultaneous administration of i) the EGFR TKI and ii) Smac mimetic to the human patient. In embodiments, the EGFR TKI is osimertinib or a pharmaceutically acceptable salt thereof. In further embodiments, the human patient is an EGFR TKI-naïve human patient. In further embodiments, the human patient has previously received EGFR TKI treatment. In further embodiments, the human patient has previously received osimertinib or a pharmaceutically acceptable salt thereof. In still further embodiments, the cancer is lung cancer, such as NSCLC. In yet further embodiments, the NSCLC is an EGFR mutation-positive NSCLC.

In an aspect there is provided Smac mimetic for use in the treatment of cancer in a human patient, wherein the treatment comprises the separate, sequential, or simultaneous administration of i) an EGFR TKI and ii) the Smac mimetic to the human patient. In embodiments, the EGFR TKI is osimertinib or a pharmaceutically acceptable salt thereof. In further embodiments, the human patient is an EGFR TKI-naïve human patient. In further embodiments, the human patient has previously received EGFR TKI treatment. In further embodiments, the human patient has previously received osimertinib or a pharmaceutically acceptable salt thereof. In still further embodiments, the cancer is lung cancer, such as NSCLC. In yet further embodiments, the NSCLC is an EGFR mutation-positive NSCLC.

In an aspect there is provided a method of treating cancer in a human patient in need of such a treatment comprising administering to the human patient a therapeutically effective amount of a Smac mimetic, wherein the treatment comprises the separate, sequential, or simultaneous administration of i) a therapeutically effective amount of an EGFR TKI and ii) a therapeutically effective amount of the Smac mimetic to the human patient. In embodiments, the EGFR TKI is osimertinib or a pharmaceutically acceptable salt thereof. In further embodiments, the human patient is an EGFR TKI-naïve human patient. In further embodiments, the human patient has previously received EGFR TKI treatment. In further embodiments, the human patient has previously received osimertinib or a pharmaceutically acceptable salt thereof. In still further embodiments, the cancer is lung cancer, such as NSCLC. In yet further embodiments, the NSCLC is an EGFR mutation-positive NSCLC.

In an aspect there is provided use of a Smac mimetic in the manufacture of a medicament for the treatment of cancer in a human patient, wherein the treatment comprises the separate, sequential, or simultaneous administration of i) an EGFR TKI and ii) the Smac mimetic to the human patient. In embodiments, the EGFR TKI is osimertinib or a pharmaceutically acceptable salt thereof. In further embodiments, the human patient is an EGFR TKI-naïve human patient. In further embodiments, the human patient has previously received EGFR TKI treatment. In further embodiments, the human patient has previously received osimertinib or a pharmaceutically acceptable salt thereof. In still further embodiments, the cancer is lung cancer, such as NSCLC. In yet further embodiments, the NSCLC is an EGFR mutation-positive NSCLC.

In an aspect there is provided a kit comprising:
a first pharmaceutical composition comprising an EGFR TKI and a pharmaceutically acceptable diluent or carrier; and
a second pharmaceutical composition comprising a Smac mimetic and a pharmaceutically acceptable diluent or carrier.

In an aspect, there is provided a Smac mimetic for use in the treatment of non-small cell lung cancer in a human patient, wherein the patient's disease has reached maximal response during or after previous EGFR TKI treatment. In embodiments, the human patient's disease has progressed during or after previous treatment with osimertinib or a pharmaceutically acceptable salt thereof. In embodiments, the treatment with a Smac mimetic induces cell death in drug tolerant persister cells.

In one aspect, there is provided osimertinib or a pharmaceutically acceptable salt thereof in the treatment of non-small cell lung cancer in a human patient, wherein the human patient's disease has progressed during or after previous treatment with a different EGFR TKI.

In an aspect, there is provided a method of treating non-small cell lung cancer in a human patient in need of such a treatment comprising administering to the human patient a therapeutically effective amount of a Smac mimetic, wherein the patient's disease has progressed during or after previous EGFR TKI treatment. In embodiments, the human patient's disease has progressed during or after previous treatment with osimertinib or a pharmaceutically acceptable salt thereof. In embodiments, the treatment with a Smac mimetic induces cell death in drug tolerant persister cells.

In an aspect, there is provided the use of a Smac mimetic in the manufacture of a medicament for the treatment of non-small cell lung cancer in a human patient, wherein the patient's disease has progressed during or after previous EGFR TKI treatment. In embodiments, the human patient's disease has progressed during or after previous treatment with osimertinib or a pharmaceutically acceptable salt thereof. In embodiments, the treatment with a Smac mimetic induces cell death in drug tolerant persister cells.

### EXAMPLES

The specific Examples below, with reference to the accompanying Figures, are provided for illustrative purposes only and are not to be construed as limiting the teachings herein.

PC9 is a cell line derived from human lung adenocarcinoma harbouring the activating mutation in EGFR del E746_A750 (Ex19-del). HCC2935 is a cell line derived from a pleural effusion of human lung adenocarcinoma harbouring the activating mutation in EGFR del E746_T751 (Ex19-del). HCC2279 is a cell line derived from a human lung adenocarcinoma bearing the activating mutation in EGFR del Em746_A750. HCC4006 is a cell line derived from a human lung adenocarcinoma bearing the activating mutation in EGFR del E746_A750. II-18 is a cell line derived from a human lung adenocarcinoma bearing the activating mutation in EGFR L858R. NCI-H1975 is a cell line derived from a human lung adenocarcinoma bearing the activating mutation in EGFR L858R and the gatekeeper mutation in EGFR T790M.

Unless otherwise stated, all reagents are commercially available and were used as supplied.

### Example 1: A Subset of EGFRm Cell Lines Show Upregulation of c-IAP1 and c-IAP2 After Prolonged Osimertinib Treatment In Vitro

The purpose of this experiment was to use RNAseq to analyse gene expression in EGFRm cell lines treated both chronically (14 d) or acutely (24 h) with osimertinib. The data demonstrate that the mRNAs coding for c-IAP1 and c-IAP2 (BIRC2 and BIRC3, respectively) are significantly upregulated in PC9, HCC2935 and NCI-H1975 cell lines after osimertinib treatment, particularly the chronic (DTP) schedule.

### Example 2. Combination Treatment with Osimertinib+Smac Mimetics Enhances the Apoptotic Response in EGFRm Cell Lines Compared to Osimertinib Alone In Vitro

The purpose of this experiment was to show that the induction of apoptotic cell death by osimertinib could be increased by the addition of a Smac mimetic. The data demonstrate that this effect was achieved because for each of the cell lines in the panel, the number of apoptotic events (normalised to cell confluence) in the combination treated group was significantly higher than what was seen in the osimertinib monotherapy group.

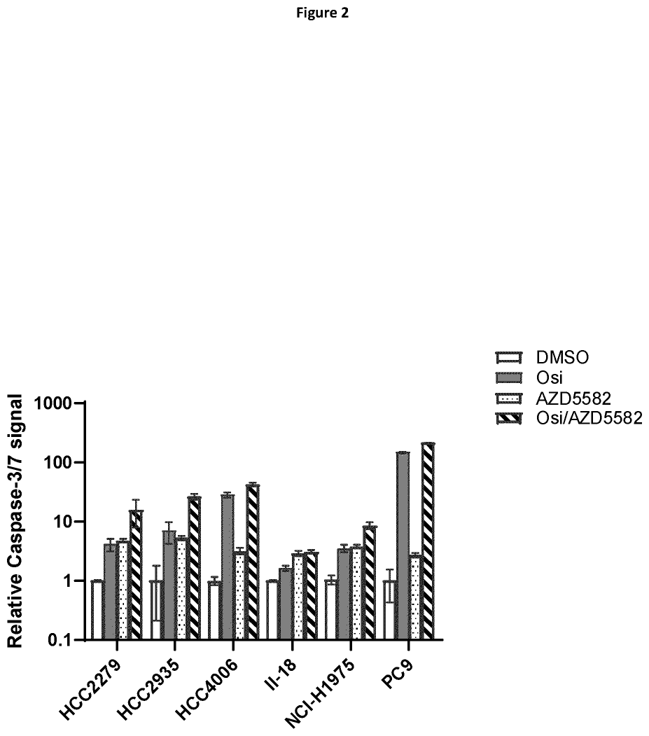

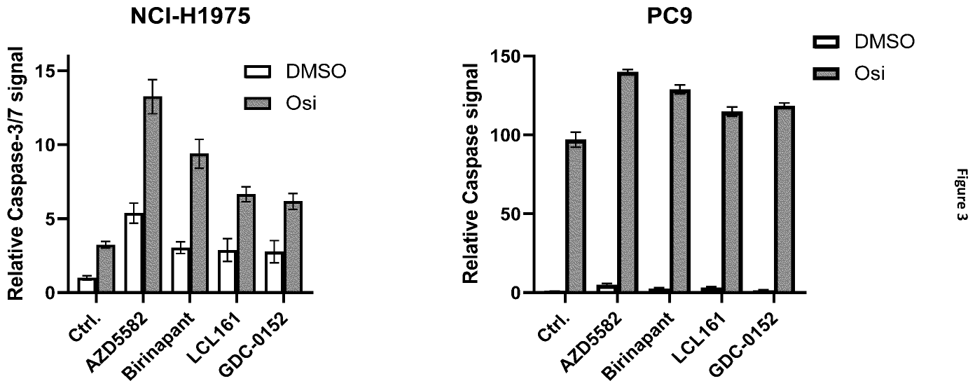

EGFRm parental cells (HCC2279, HCC2935, HCC4006, II-18, NCI-H1975 and PC9) were seeded in 96 well plated at a concentration of 5000 cells/well. The next day, cells were treated with osimertinib monotherapy (160 nM), Smac mimetic monotherapy (1 μM) or the combination thereof as well as the Incucyte Caspase 3/7 reagent (green) at a final concentration of 1 μM. Cells were then placed on the Incucyte S3 imaging system and both cell confluence and green fluorescence were measured, every 4 hours. After 96 h the experiment was terminated, and apoptosis values calculated by dividing the number of individual green points (apoptosis events) by cell confluence. For each cell line, data was normalized to DMSO treated values at 48 h (peak apoptosis). The data for all 6 cell lines treated with osimertinib+AZD5582 are shown in FIG. 2. The data for PC9 and NCI-H1975 cell lines treated with osimertinib+4 distinct Smac mimetic compounds are shown in FIG. 3.

### Example 3. Combination Treatment with Osimertinib and Smac Mimetic Compounds Inhibits the Formation of Osimertinib Drug Tolerant Persister Cells, and Smac Mimetic Monotherapy Inhibits the Regrowth of Established Persister Cells In Vitro

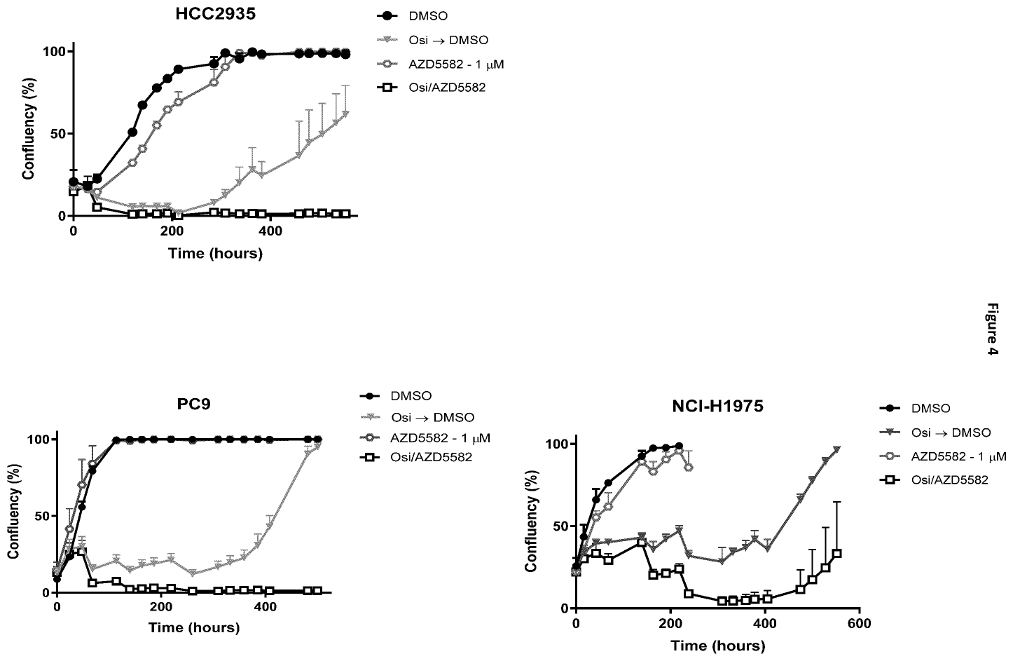

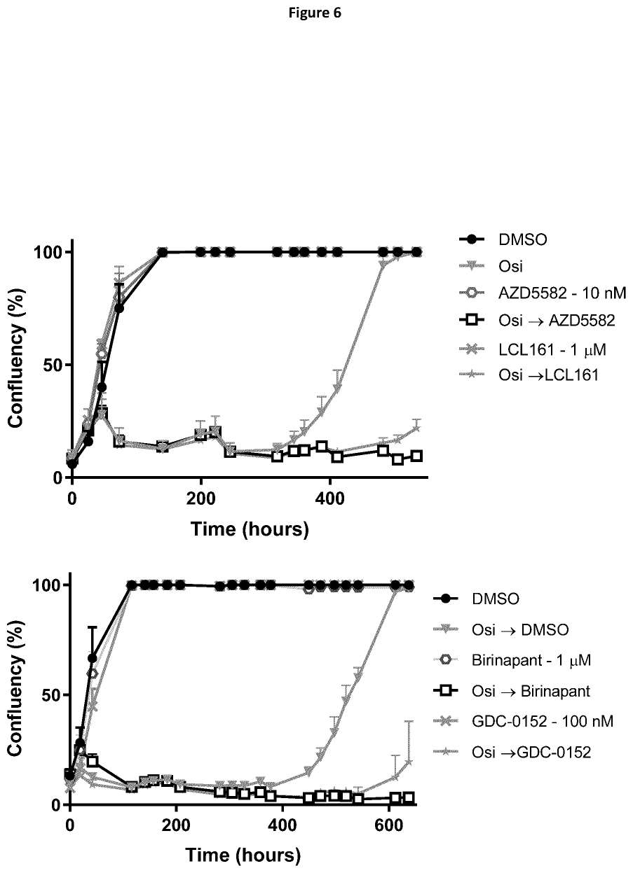

The purpose of this experiment was to show that treatment with a Smac mimetic inhibits the establishment of drug-tolerant persister cells after EGFR TKI treatment and inhibits the re-growth of persister cells after EGFR TKI monotherapy. The data demonstrate that this effect was achieved because PC9, HCC2935 or NCI-H1975 cells treated for 10 days with a combination of osimertinib and AZD5582 showed a lower percentage confluency (a measure of cell growth) at the end of the experiment than cells treated for 10 days with osimertinib alone (FIG. 4). Similarly, PC9 cells treated for 10 days with osimertinib followed by treatment with 4 distinct Smac mimetic molecules showed a lower percentage confluency (a measure of cell growth) at the end of the experiment compared with osimertinib alone without subsequent treatment with Smac mimetics (FIG. 6).

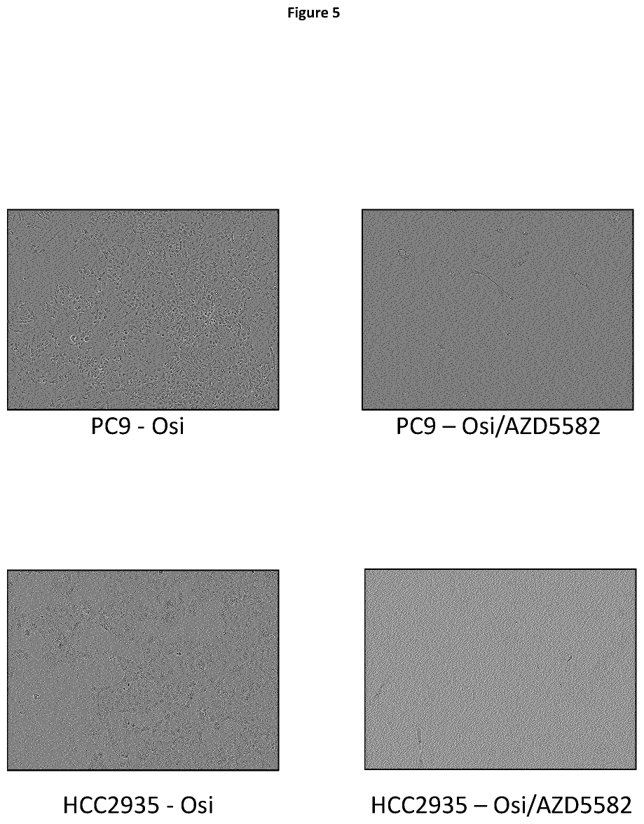

Cells were plated in 48 well plates at a concentration of 40,000 cells/well. The following day cells were treated with either osimertinib monotherapy (500 nM), the indicated doses of a Smac mimetic the combination of the two agents, and confluence measurement was begun using the Incucyte imaging platform. After 10 days, combination treated wells, as well as one subset of osimertinib monotherapy wells, were washed 2× with phosphate-buffered saline (PBS) and replaced with drug-free media. In a separate experiment, PC9 cells were treated with osimertinib monotherapy for 10 days, washed 2× with PBS and replaced with media containing the indicated doses of a Smac mimetic, or control media (DMSO). Confluence measurements continued for a further 12-17 days, and results were plotted using PRISM software. The data are shown in FIGS. 4, 5 and 6.

### Example 4. Smac Mimetic Treatment Induces Apoptosis in Osimertinib Drug-Tolerant Persister Cells In Vitro

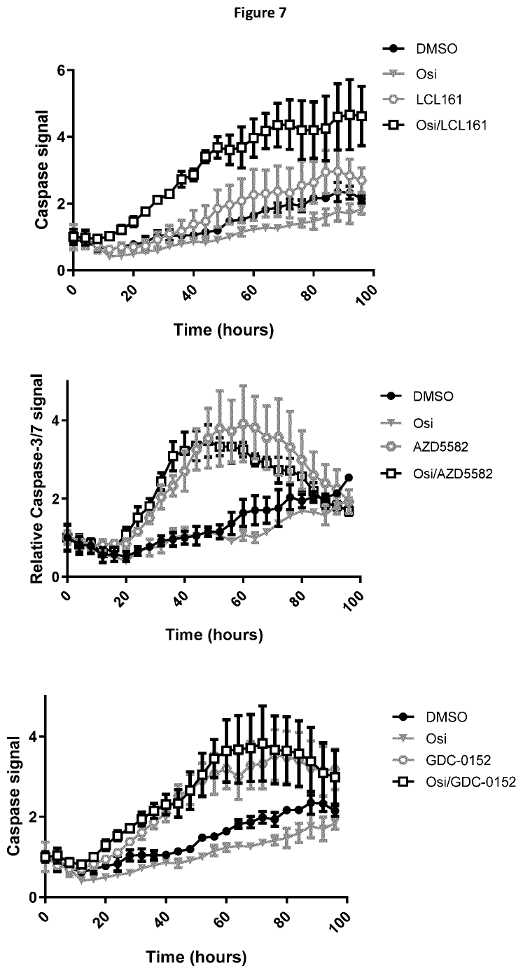

The purpose of this experiment was to show that treatment with a Smac mimetic induces apoptosis in osimertinib drug-tolerant persister (DTP) PC9 cells. The data demonstrate that this effect was achieved because enhanced caspase activity (an indicator of apoptosis) was observed in DTP cells treated with Smac mimetic monotherapy, or a combination of osimertinib and Smac mimetic when compared to DTP cells treated with control media (DMSO) or osimertinib alone (FIG. 7).

PC9 parental cells were treated for 10 days with 500 nM osimertinib to establish drug-tolerant persister cells. At this time, cells were treated with 1 μM dose of the indicated Smac mimetic+/−osimertinib (500 nM), continued osimertinib monotherapy (500 nM), or control drug-free media. All wells were additionally treated with Incucyte Caspase 3/7 reagent (1 μM). Cells were then placed on the Incucyte S3 imaging system and both cell confluence and green fluorescence were measured, every 4 hours. After 96 h the experiment was terminated, and apoptosis values calculated by dividing the number of individual green points (apoptosis events) by cell confluence. For each treatment, data was normalized to osimertinib monotherapy treated values at time 0. The data are shown in FIG. 6.

### Example 5. The Smac Mimetic Inhibitor AZD5582 Enhances the Antiproliferative Effects of Osimertinib in PC9 Xenograft In Vivo

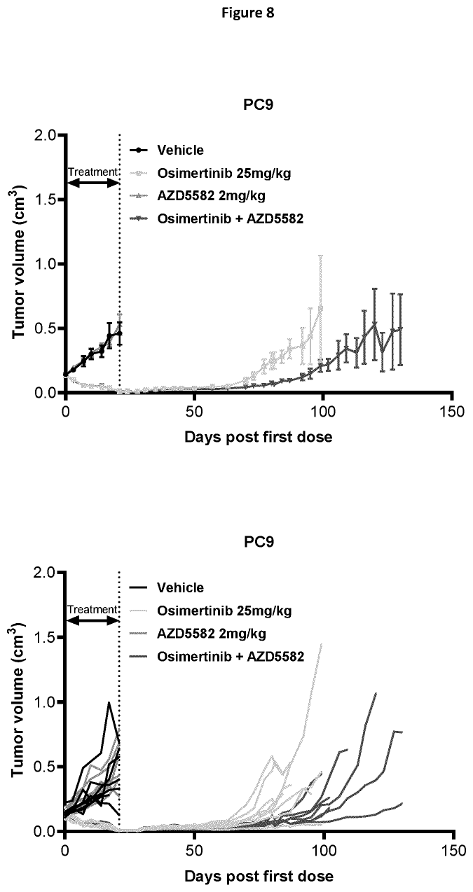

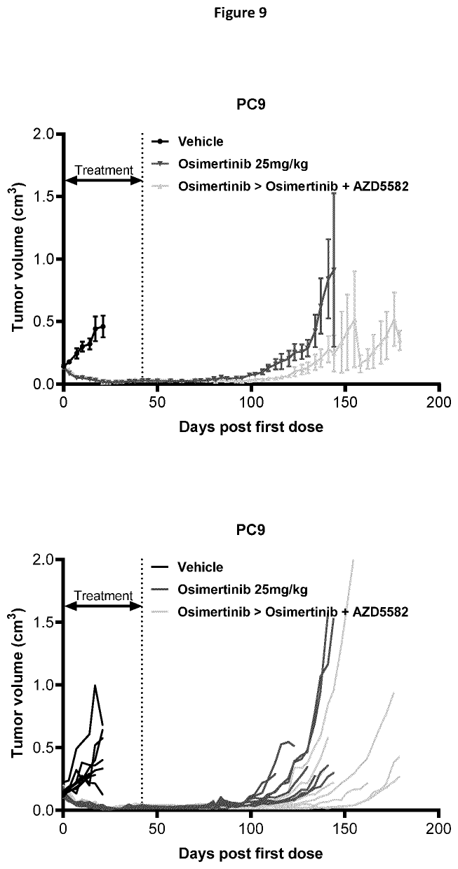

The purpose of this experiment was to show that treatment with a Smac mimetic enhances the anti-tumor effect of an EGFR TKI treatment and delays the re-growth after treatment release in vivo. The data demonstrate that this effect was achieved because PC9 xenografts treated for 21 days with a combination of osimertinib and AZD5582 showed a delay of regrowth than cells treated for 21 days with osimertinib alone (FIG. 8). Similarly, PC9 xenografts treated for 21 days with osimertinib followed by treatment for 21 days with the combination of AZD5582 and osimertinib showed a delay of regrowth when compared with PC9 xenograft treated for 42 days with osimertinib alone without subsequent treatment with Smac mimetics (FIG. 9).

1. An EGFR TKI for use in the treatment of cancer in a human patient, wherein the EGFR TKI is administered in combination with a Smac mimetic.

2. An EGFR TKI for use as claimed in claim 1, where the administration of the EGFR TKI and the Smac mimetic is separate, sequential, or simultaneous.

3. An EGFR TKI for use as claimed in claim 2, where the administration of the EGFR TKI and the Smac mimetic is sequential and the EGFR TKI is administered prior to the Smac mimetic.

4. An EGFR TKI for use as claimed in any of the previous claims, wherein the EGFR TKI is selected from the group consisting of osimertinib or a pharmaceutically acceptable salt thereof, AZD3759 or a pharmaceutically acceptable salt thereof, lazertinib or a pharmaceutically acceptable salt thereof, abivertinib or a pharmaceutically acceptable salt thereof, alflutinib or a pharmaceutically acceptable salt thereof, afatinib or a pharmaceutically acceptable salt thereof, CX-101 or a pharmaceutically acceptable salt thereof, HS-10296 or a pharmaceutically acceptable salt thereof, BPI-7711 or a pharmaceutically acceptable salt thereof, dacomitinib or a pharmaceutically acceptable salt thereof, icotinib or a pharmaceutically acceptable salt thereof, gefitinib or a pharmaceutically acceptable salt thereof and erlotinib or a pharmaceutically acceptable salt thereof.

5. An EGFR TKI for use as claimed in any of the previous claims, wherein the EGFR TKI is selected from the group consisting of osimertinib or a pharmaceutically acceptable salt thereof, AZD3759 or a pharmaceutically acceptable salt thereof, alflutinib or a pharmaceutically acceptable salt thereof, HS-10296 or a pharmaceutically acceptable salt thereof, and lazertinib or a pharmaceutically acceptable salt thereof.

6. An EGFR TKI for use as claimed in any of the previous claims, wherein the EGFR TKI is osimertinib or a pharmaceutically acceptable salt thereof.

7. An EGFR TKI for use as claimed in any of the previous claims, wherein the Smac mimetic is selected from the group consisting of AZD5582 or a pharmaceutically acceptable salt thereof, birinapant or a pharmaceutically acceptable salt thereof, LCL161 or a pharmaceutically acceptable salt thereof, GDC-0152 or a pharmaceutically acceptable salt thereof, GDC-0917 or a pharmaceutically acceptable salt thereof HGS1029 or a pharmaceutically acceptable salt thereof and AT-406 or a pharmaceutically acceptable salt thereof.

8. An EGFR TKI for use as claimed in any of the previous claims, wherein the cancer is non-small cell lung cancer.

9. An EGFR TKI for use as claimed in claim 8, wherein the non-small cell lung cancer is an EGFR mutation-positive non-small cell lung cancer.

10. An EGFR TKI for use as claimed in claim 9, wherein the EGFR mutation-positive non-small cell lung cancer comprises activating mutations in EGFR selected from exon 19 deletions and L858R substitution mutations.

11. An EGFR TKI for use as claimed in claim 9 or claim 10, wherein the EGFR mutation-positive non-small cell lung cancer comprises a T790M mutation.

12. An EGFR TKI for use as claimed in any one of claims 1 to 10, wherein the human patient is an EGFR TKI-naïve human patient.

13. An EGFR TKI for use as claimed in any one of claims 1 to 11, wherein the human patient's disease has progressed during or after previous EGFR TKI treatment.

14. An EGFR TKI for use for use as claimed in claim 13, wherein the EGFR TKI is Osimertinib or a pharmaceutically acceptable salt thereof and the human patient's disease has progressed during or after previous treatment with a different EGFR TKI.

15. An EGFR TKI for use as claimed in any of the previous claims, wherein the cancer upregulates IAP.

16. The use of an EGFR TKI in the manufacture of a medicament for the treatment of cancer in a human patient, wherein the EGFR TKI is administered in combination with a Smac mimetic.

17. A method of treating cancer in a human patient in need of such a treatment, comprising administering to the human patient a therapeutically effective amount of an EGFR TKI, wherein the EGFR TKI is administered in combination with a therapeutically effective amount of a Smac mimetic.

18. A pharmaceutical composition comprising an EGFR TKI, a Smac mimetic and a pharmaceutically acceptable diluent or carrier.

19. A Smac mimetic for use in the treatment of non-small cell lung cancer in a human patient, wherein the patient's disease has reached maximal response during or after previous EGFR TKI treatment.

20. A Smac mimetic for use in the treatment of non-small cell lung cancer as claimed in claim 18, where the EGFR TKI is osimertinib or a pharmaceutically acceptable salt thereof.
